(19) United States
(12) Patent Application Publication
(10) Pub. No.: US 2026/0053563 A1
(43) Pub. Date: Feb. 26, 2026

(54) APPARATUS FOR IMAGING AND TREATMENT OF SKIN OF SUBJECT

(71) Applicant: LUMENIS BE LTD., Yokneam (IL)

(72) Inventors: Andrey Gandman, Haifa (IL); Albert Ben Shlomo, Netanya (IL)

(21) Appl. No.: 19/374,165

(22) Filed: Oct. 30, 2025

Related U.S. Application Data
(63) Continuation of application No. PCT/IL2024/050418, filed on May 2, 2024.
(63) Continuation-in-part of application No. 17/226,235, filed on Apr. 9, 2021.
(60) Provisional application No. 63/463,926, filed on May 4, 2023.

(51) Int. Cl. A61B 18/20; A61B 18/00

(52) U.S. Cl. CPC A61B 18/203; A61B 2018/00184; A61B 2018/00452; A61B 2018/20355; A61B 2018/20553; A61B 2018/205545

(57) ABSTRACT

An apparatus for imaging and treatment of skin of a subject, comprising a frame configured to circumscribe a target region of the skin. The apparatus further comprises an imaging branch and a laser branch contained in the frame. The imaging branch has an imaging path and configured to generate an image of the target region of the skin that is in proximity to a first end of the frame. The laser branch has a laser path and is configured to radiate electromagnetic rays from a second end of the frame towards the first end for treatment of the target region of the skin. The laser branch is adapted to radiate a circular beam of electromagnetic rays at the second end of the frame, and output a polygonal beam of electromagnetic rays at the first end of the frame for treatment of the target region of the skin of the subject.

### RELATED APPLICATIONS

This application is a continuation-in-part of U.S. application Ser. No. 17/226,235, filed Apr. 9, 2021 and is also a continuation of PCT Application No. PCT/IL2024/050418, filed May 2, 2024 which claims priority to U.S. Provisional Application No. 63/463,926 filed May 4, 2023, entitled “Apparatus for Imaging and Treatment of Skin of Subject” the entire contents of which are herein incorporated by reference.

### TECHNICAL FIELD

The present disclosure relates to the field of medical devices. Particularly, the present disclosure relates to an apparatus for imaging and treatment of skin of a subject.

### BACKGROUND

Therapeutic and aesthetic energy-based treatments, such as lasers, are utilized for procedures on skin, such as hair removal, tattoo removal, vascular removal, pigmented lesions, skin tightening, and/or skin rejuvenation.

Typically, medical personnel manually use a handpiece to deliver such treatments, and the medical personnel will assess skin attributes to determine the laser parameters for treatment. The skin attributes may be skin type, presence of tanning, hair color, hair density, hair thickness, blood vessel diameter, blood vessel depth, lesion type, pigment depth, pigment intensity, tattoo color, tattoo type.

PCT publication number WO2020075162A1, filed 7 Oct. 2019, assigned to the assignee of the present disclosure, is directed to some features of the therapeutic and aesthetic energy-based treatment, and is herein incorporated by reference in its entirety. U.S. application Ser. No. 17/565,709 filed 30 Dec. 2021, assigned to the assignee of the present disclosure, is directed to some features of a therapeutic and aesthetic energy-based treatment, and is herein incorporated by reference in its entirety.

### SUMMARY

The present disclosure advances the state-of-the-art technology for sensing and analyzing skin condition by, for example, generating an image of the skin to be treated, which can be especially advantageous for formulating a skin treatment regimen, and performing the skin treatment regimen.

Pursuant to an aspect of the present disclosure, in a non-limiting embodiment, an apparatus for imaging and treatment of skin of a subject is disclosed. The apparatus comprises a frame configured to circumscribe a target region of the skin of the subject. The frame has a first end and a second end opposite to the first end. The apparatus further comprises an imaging branch contained in the frame. The imaging branch has an imaging path and is configured to generate an image of the target region of the skin that is in proximity to the first end of the frame. The apparatus furthermore comprises a laser branch contained in the frame. The laser branch has a laser path and is configured to radiate electromagnetic rays from the second end of the frame towards the first end of the frame for treatment of the target region of the skin. The laser branch is adapted to radiate a circular beam of electromagnetic rays at the second end of the frame, and output a beam of electromagnetic rays having a polygonal shape, such as a rectangle or hexagon, at the first end of the frame for treatment of the target region of the skin of the subject.

In another non-limiting embodiment of the present disclosure, the laser branch is configured to generate a polygonal (e.g., rectangular, hexagonal) treatment spot based on the polygonal beam of electromagnetic rays.

In another non-limiting embodiment of the present disclosure, the laser branch is configured to generate the polygonal treatment spot having varying size.

In another non-limiting embodiment of the present disclosure, the laser branch is configured to generate the polygonal treatment spot having a rectangular, specifically square, shape.

In another non-limiting embodiment of the present disclosure, the laser branch is configured to generate the polygonal treatment spot having a hexagonal shape.

In another non-limiting embodiment of the present disclosure, the imaging path and the laser path of the apparatus have a common path at the first end of the frame.

In another non-limiting embodiment of the present disclosure, the laser branch comprises a fiber connection/guide disposed at the second end of the frame. The fiber guide is configured to radiate the circular beam of electromagnetic rays. The laser branch further comprises a laser lens assembly disposed downstream of the fiber guide in a direction of electromagnetic rays. The laser lens assembly is adapted to convert the circular beam of electromagnetic rays into the polygonal beam of electromagnetic rays.

In another non-limiting embodiment of the present disclosure, the laser branch comprises a beam combiner disposed downstream of the laser lens assembly in the direction of electromagnetic rays. The beam combiner is configured to project the polygonal beam of electromagnetic rays on the target region of the skin of the subject.

In another non-limiting embodiment of the present disclosure, the laser lens assembly comprises a static lens unit and a dynamic lens unit disposed upstream of the static lens unit in the direction of electromagnetic rays. The dynamic lens unit is configured to move relative to the static lens unit along the direction of electromagnetic rays. In some embodiments, the laser lens assembly further comprises a motor and a lens motion assembly, which may be a nut-bracket assembly, operatively coupled with the dynamic lens unit for moving the dynamic lens unit relative to the static lens unit.

In another non-limiting embodiment of the present disclosure, the laser branch comprises a beam collimation assembly arranged between the fiber guide and the laser lens assembly. The beam collimation assembly is adapted to collimate and project the circular beam of electromagnetic rays on the laser lens assembly.

In another non-limiting embodiment of the present disclosure, the imaging branch comprises a first mirror arranged parallel to the beam combiner. The beam combiner is adapted to collect backscattered illumination light from the target region of the skin and reflect the collected illumination light on the first mirror. The imaging branch further comprises an image lens assembly disposed downstream of the first mirror in a direction of collected illumination light. The image lens assembly is adapted to converge the collected illumination light reflected from the first mirror. The image lens assembly furthermore comprises a second mirror arranged parallel to the first mirror and downstream of the image lens assembly in the direction of collected illumination light. The second mirror is adapted to reflect the converged collected illumination light towards an image detector assembly.

In another non-limiting embodiment of the present disclosure, the imaging branch comprises a polarizer unit disposed between the second mirror and the image detector assembly for polarizing the collected illumination light.

In another non-limiting embodiment of the present disclosure, the image lens assembly comprises a plurality of lenses, and an aperture arranged between the plurality of lenses for converging the collected illumination light.

In another non-limiting embodiment of the present disclosure, the beam combiner, the first mirror and the second mirror are arranged inclined with respect to the direction of electromagnetic rays and/or the direction of collected illumination light.

In another non-limiting embodiment of the present disclosure, the beam combiner is configured to refract the electromagnetic rays and reflect the collected illumination light.

In another non-limiting embodiment of the present disclosure, the apparatus comprises a light emitting diode (LED) assembly disposed at the first end of the frame. The LED assembly is configured to illuminate the target region of the skin of the subject with illumination light.

In another non-limiting embodiment of the present disclosure, the frame comprises a sapphire window assembly at the first end thereof. The sapphire window assembly is adapted to receive backscattered illumination light from the target region of the skin in the imaging path, and project the polygonal beam of electromagnetic rays on the target region of the skin in the laser path.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The novel features and characteristics of the disclosure are set forth in the description. The disclosure itself, however, as well as a preferred mode of use, further objectives, and advantages thereof, will best be understood by reference to the following description of an illustrative embodiment when read in conjunction with the accompanying drawings. One or more embodiments are now described, by way of example only, with reference to the accompanying drawings wherein like reference numerals represent like elements and in which:

FIG. 1 is a side cross-sectional view of an apparatus for imaging and treatment of skin of a subject, the apparatus comprising an imaging branch and a laser branch, in accordance with some embodiments of the present disclosure;

FIG. 2 is another side cross-sectional view of the apparatus of FIG. 1, depicting flow of rays in the imaging branch and the laser branch, in accordance with some embodiments of the present disclosure;

FIG. 3 is a schematic ray diagram of the laser branch of the apparatus of FIG. 1, in accordance with some embodiments of the present disclosure; and

FIG. 4 is a schematic ray diagram of the imaging branch of the apparatus of FIG. 1, in accordance with some embodiments of the present disclosure.

Skilled artisans will appreciate that elements in the drawings are illustrated for simplicity and have not necessarily been drawn to scale. For example, the dimensions of some of the elements in the drawings may be exaggerated relative to other elements to help to improve understanding of embodiments of the present disclosure.

### DETAILED DESCRIPTION

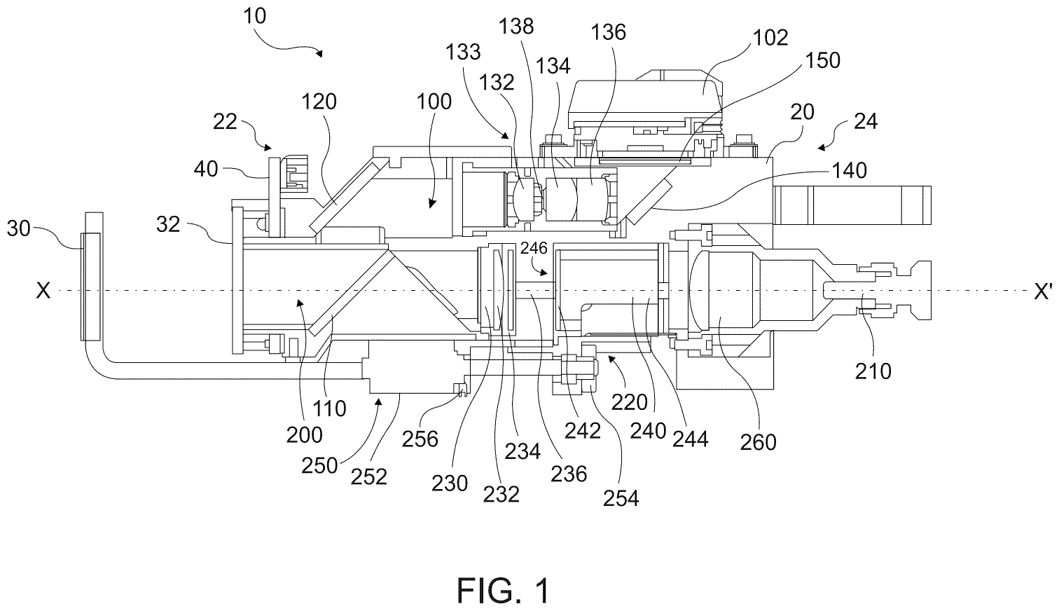

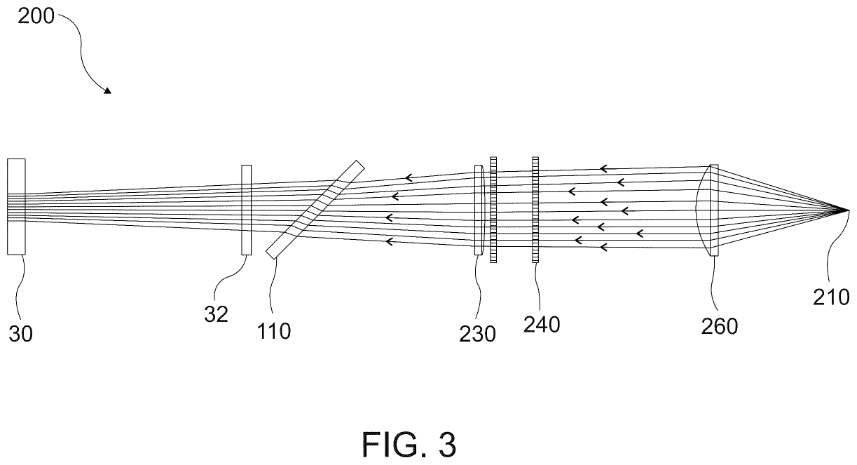

While the disclosure is susceptible to various modifications and alternative forms, specific embodiments thereof have been shown by way of example in FIGS. 1 to 4 and will be described in detail below. It should be understood, however, that it is not intended to limit the disclosure to the particular forms disclosed, but on the contrary, the disclosure is to cover all modifications, equivalents, and alternatives falling within the scope of the disclosure as defined by the appended claims. Additional features and advantages are realized through the techniques of the present disclosure. Other embodiments and aspects of the disclosure are described in detail herein and are considered a part of the claimed disclosure.

Before describing detailed embodiments, it may be observed that the present disclosure is directed to an apparatus for imaging and treatment of skin of a subject. It is to be noted that a person skilled in the art can be motivated from the present disclosure and modify the various constructions of the apparatus. However, such modifications should be construed some embodiments. Accordingly, the drawings are showing only those specific details that are pertinent to understanding the embodiments of the present disclosure so as not to obscure the disclosure with details that will be readily apparent to those of ordinary skill in the art having benefit of the description herein.

In the present disclosure, the term “exemplary” is used herein to mean “serving as an example, instance, or illustration.” Any embodiment or implementation of the present subject matter described herein as “exemplary” is not necessarily to be construed as preferred or advantageous over other embodiments.

The terms “comprises”, “comprising”, or any other variations thereof, are intended to cover non-exclusive inclusions, such that a device that comprises a list of components does not include only those components but may include other components not expressly listed or inherent to such device. In other words, one or more elements in a system or apparatus proceeded by “comprises . . . a” does not, without more constraints, preclude the existence of other elements or additional elements in the system or apparatus.

The terms like “at least one” and “one or more” may be used interchangeably or in combination throughout the description.

Reference will now be made to the exemplary embodiments of the disclosure, as illustrated in the accompanying drawings. Wherever possible same numerals will be used to refer to the same or like parts.

Aspects of the disclosure are described in the following paragraphs with reference to FIGS. 1 to 4. In FIGS. 1 to 4, the same element or elements which have same functions are indicated by the same reference signs.

In some embodiments of the present disclosure, there is an apparatus (10) for imaging and treatment of skin of a subject. In some embodiments, the apparatus (10) of the present disclosure is configured for imaging and treatment of humans. In some embodiments, the apparatus (10) may be configured for imaging and treatment of non-human animals. Specifically, the apparatus (10) may be configured for imaging and real time, and simultaneous or sequential, laser treatment of a target region of the skin of a human. In some embodiments, the laser used for laser treatments is an Yttrium Aluminum Garnet; Y₃Al₅O₁₂ (YAG) laser. In some embodiments, any laser normally employed for treatment of skin may be used with parallel changes to optic size and lens coatings of the apparatus.

The apparatus (10) may comprise a treatment laser branch that may be one that targets the skin tissue, the rays get absorbed by one or more chromophores and causes a cascade of reactions, including one or more of photochemical, photothermal, thermal, photoacoustic, acoustic, healing, ablation, coagulation, biological, tightening, or other any other physiological effect. Those reactions create the desired treatment outcomes such as permanent hair removal, hair growth, pigmented or vascular lesion treatment of soft tissue, rejuvenation or tightening, acne treatment, cellulite treatment, vein collapse, or tattoo removal which may include mechanical breakdown of tattoo pigments and crusting.

Skin tissue is a very complex biological organ. Although the basic structure is common to all humans, there are many variations within the different areas in a specific individual and among individuals. Variations include skin color (melanin content in Basal layer), hair color and thickness, collagen integrity, blood vessel structure, vascular and pigmented lesions of various types, foreign objects like tattoos, etc.

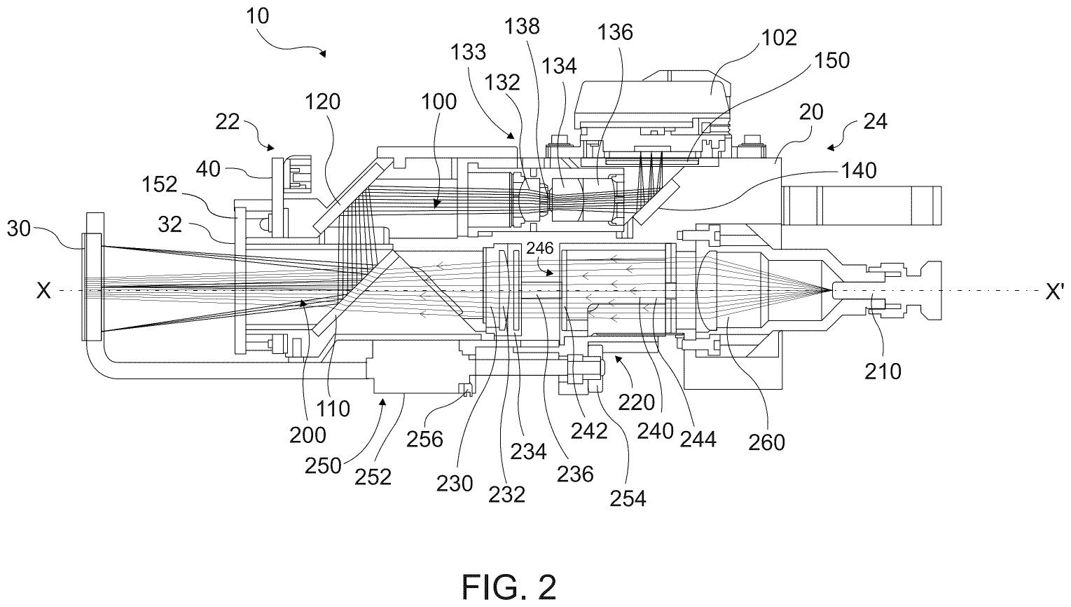

Referring to FIGS. 1 and 2, side cross-sectional views of the apparatus (10) for imaging and treatment of the target region of the skin of the subject are illustrated. In some embodiments, the apparatus (10) comprises a frame (20) having a first end (22) and a second end (24) opposite to the first end (22) along a longitudinal axis (X-X′) of the apparatus (10). In some embodiments, the frame (20) of the apparatus (10) may be embodied as a handpiece having a handle and/or a gripping surface such that the apparatus (10) can be held, like a pen, by a medical practitioner including doctor/surgeon/therapist.

In some embodiments, the frame (20) is configured to circumscribe the target region of the skin of the subject. In some embodiments, circumscribing the target region of the skin comprises confining at least a portion of the skin to be treated. In some embodiments, the frame (20) circumscribes the target region of the skin in order to stretch or flatten the target region for obtaining images of the target region of the skin.

In some embodiments, the frame (20) comprises a sapphire window assembly (30) at the first end (22) of the frame (20). The sapphire window assembly is optional, that is, not required for the apparatus to work. In some embodiments, the sapphire is employed for the conduction of heat away from the skin. The sapphire window assembly (30) may be held in close proximity to the target region of the skin in order to circumscribe the target region of the skin. In some embodiments, the apparatus (10) comprises an imaging branch (100) and a laser branch (200) are contained within the frame (20) of the apparatus (10). In some embodiments, the imaging branch (100) has an imaging path that extends from the first end (22) of the frame (20). The imaging branch (100) may be adapted to generate an image of the target region of the skin of the subject. Details regarding generating the image of the target region of the skin are described in the subsequent paragraphs. Further, and in some embodiments, the laser branch (200) has a laser path that extends from the second end (24) of the frame (20) to the first end (22) of the frame (20). In some embodiments, the laser branch (200) is adapted to treat the target region of the skin, using electromagnetic rays radiated by a laser source that may or may not be included within the apparatus (10). Details regarding treatment of the target region of the skin are also described in the subsequent paragraphs. In some embodiments, the laser source is included in the apparatus. In some embodiments, the laser source is a laser transmission assembly (not shown) in a handpiece of the apparatus connectable to the laser source outside of the apparatus.

In some embodiments, not specifically shown in the figure, the apparatus includes a thermoelectric cooling device (TEC) configured to maintain a working temperature range. In some embodiments, the thermoelectric cooling device may be located at the first end (22) of the frame (20). In some embodiments, the TEC cools the frame (20), which cools the sapphire window assembly (30). The TEC may be cooled using a water line provided at one side of the TEC.

In some embodiments of the present disclosure, the imaging path of the imaging branch (100) and the laser path of the laser branch (200) have a common path, as illustrated in FIGS. 1 and 2, at the first end (22) of the frame (20) of the apparatus (10). Further, in some embodiments, a longitudinal axis of the imaging branch (100) of the apparatus (10) in a direction towards the second end (24) of the frame (20) is substantially parallel to a longitudinal axis of the laser branch (200) of the apparatus (10) at the second end (24) of the frame (20), as illustrated in FIGS. 1 and 2. In some embodiments, the sapphire window assembly (30) of the frame (20) forms part of the imaging branch (100) as well as the laser branch (200) of the apparatus (10). Still referring to FIGS. 1 and 2, the frame (20) may be embodied as a hollow cuboidal structure that is adapted to house the imaging branch (100) and the laser branch (200) of the apparatus (10). The frame (20) may comprise a protective window (32) at the first end (22) of the frame (20). The protective window (32) may be formed of a glass and may be configured to allow illumination light/visible rays/electromagnetic rays to enter and exit from the frame (20) of the apparatus (10). At the second end (24) of the frame (20), the apparatus (10) may comprise a laser source (source for electromagnetic rays), for example, a fiber connection/guide, that may be part of the laser branch (200) of the apparatus (10). Further, the frame (20) may comprise an aperture defined in the top side of the frame (20). The aperture may provide a seating space for an image detector assembly (102) of the imaging branch (100) of the apparatus (10).

Further, as discussed above, the imaging branch (100) may be adapted to generate the image of the target region of the skin of the subject. To aid in generating the image, the apparatus (10) may comprise a Light Emitting Diode (LED) assembly (40) at the first end (22) of the frame (20). In some embodiments, the LED assembly (40) is be disposed within the frame (20) of the apparatus (10) and behind the protective window (32) of the frame (20). In some embodiments, the LED assembly (40) is configured to illuminate the target region of the skin of the subject. The LED assembly (40) may comprise one or more LED light sources positioned around the imaging path (200). In some embodiments, the LED light sources may be symmetrically positioned with respect to the imaging path. The LED light sources may have peak wavelengths in the range of 300 nm to 1100 nm. In some embodiments, the LED assembly (40) has two red LED light sources with a peak wavelength of 660 nm, four yellow LED light sources with a peak wavelength of 590 nm, two infrared LED light sources with a peak wavelength of 860 nm, four cyan LED light sources with a peak wavelength of 490 nm, two blue LED light sources with a peak wavelength of 450 nm, and four green LED light sources with a peak wavelength of 530 nm. The number of LED light sources for each peak wavelength may be determined by the intensity of the peak wavelength required to obtain an image illuminated evenly. Additionally, the LED assembly (40) may comprise a heating system configured to maintain the temperature of the LED light sources in the range of 25 to 35 degrees Celsius, which is optimal to maintain the intensity of the LED light sources.

With reference to FIGS. 1, 2 and 4, an exemplary embodiment of the imaging branch (100) of the apparatus (10) for imaging and treatment of skin of the subject is disclosed. As discussed above, the imaging branch (100) may be contained in the frame (20) of the apparatus (10) and may be configured to generate the image of the target region of the skin that is in proximity to the first end (22) of the frame (20). Specifically, during use of the apparatus (10), the sapphire window assembly (30) of the frame (20) may be held in close proximity to the target region (to be treated) of the skin, and the image branch (100) may be configured to generate the image of the target region of the skin. Further, the image generated by the imaging branch (100) may be obtained or captured or seen at the top side of the frame (20) by way of the detector assembly (102), as shown in FIGS. 1 and 2. In some embodiments, the image generated by the imaging branch (100) is obtained or captured or seen at the second end (24) of the frame (20).

In some embodiments of the present disclosure, the imaging branch (100) of the apparatus (10) comprises a beam combiner (110), a first mirror (120), an image lens assembly (130) and a second mirror (140) for generating the image of the target region of the skin of the subject. In some embodiments, the illumination light emitted by the LED assembly (40) (or the LED light sources) of the apparatus (10) is backscattered by the target region of the skin. In some embodiments, said backscattered illumination light strikes the beam combiner (110) and the beam combiner (110) is adapted to collect the backscattered illumination light from the target region of the skin. Further, and in some embodiments, the beam combiner (110) acts as a reflective surface for the illumination light (having, for example, wavelength within the range of 300 nm to 1100 nm), the beam combiner (110) reflects the collected backscattered illumination light towards the first mirror (120) of the imaging branch (100). In some embodiments, the beam combiner (110) is arranged at an inclined angle, for example 45 degrees, relative to a direction of collected illumination light, and thus, by principle of reflectivity, the beam combiner (110) further bends the angle of reflected rays by another 45 degrees towards the first mirror (120).

Further, the first mirror (120) of the imaging branch (100) may be arranged substantially parallel to the beam combiner (110). In some embodiments, the first mirror (120) is also arranged at an inclined angle relative to the direction of collected illumination light. In some embodiments of the present disclosure, the first mirror (120) is adapted to receive the collected backscattered illumination light from the beam combiner (110) and further reflect the collected backscattered illumination light towards the image lens assembly (130).

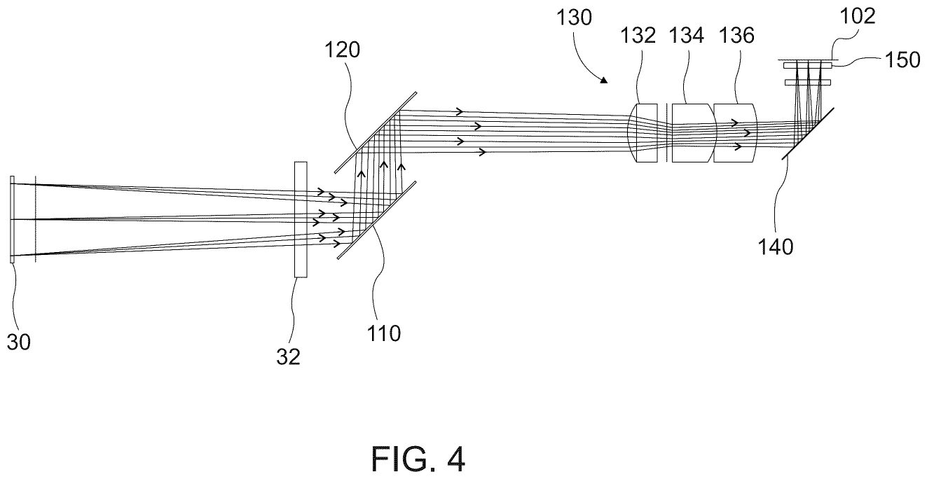

With reference to FIG. 4, and in some embodiments, the image lens assembly (130) is disposed downstream of the first mirror (120) in the direction of collected illumination light. In some embodiments, the image lens assembly (130) is configured to converge the collected scattered illumination light reflected from the first mirror (120). As illustrated in the exemplary embodiment of FIG. 4, the image lens assembly (130) comprises a plurality of lenses, for example three lenses (132, 134, 136), arranged such that the collected scattered illumination light is converged in the direction towards the second end (24) of the frame (20)/the apparatus (10). Also, the image lens assembly (130) may comprise an aperture (138) arranged between the plurality of lenses (132, 134, 136) so as to further converge and/or polarize and/or channelize the collected scattered illumination light in the direction towards the second end (24) of the frame (20).

Furthermore, and in some embodiments, the imaging branch (100) comprises the second mirror (140) disposed downstream of the image lens assembly (130) in the direction of collected illumination light. In some embodiments, the second mirror (140) is arranged substantially parallel to the first mirror (120). In some embodiments, the second mirror (140) is also arranged at an inclined angle relative to the direction of collected illumination light. In some embodiments of the present disclosure, the second mirror (140) is adapted to receive the converged collected backscattered illumination light from the image lens assembly (130) and further reflect the converged collected backscattered illumination light towards the image detector assembly (102) seated at an end of the imaging path or the imaging branch (100) of the apparatus (10), thereby generating the image of the target region of the skin at the end of the imaging branch (100) and/or the second end (24) of the frame (20) of the apparatus (10).

In some embodiments of the present disclosure, the imaging branch (100) of the apparatus (10) comprises a polarizer system configured to illuminate the skin with polarized illumination light and detect corresponding collected light. This assures that the light that is backscattered from the skin is of a diffuse (scattered) nature and helps to eliminate the specular reflections from the skin surface, such that the detected light is indicative of the deeper skin tissue layers. As shown, the apparatus may comprise a first polarizing unit (152) in the path of the illumination light towards the tissue, and a second polarizing unit (150) in the path of the collected light, disposed between the second mirror (140) and the image detector assembly (102). The first and second polarizing units may be positioned in a cross polarized configuration, i.e. with a 90° angle, therebetween. The polarizer system serves to generate a clear and refined image of the target region of the skin at the end of the imaging branch (100) or the second end (24) of the frame (20) of the apparatus (10).

Additionally, in some embodiments, the apparatus (10) may comprise one or more blackout components and blackout surfaces in the imaging branch (100) of the apparatus (10) to prevent any loss of collected backscattered illumination light in the image path of the apparatus (10).

With reference to FIGS. 1, 2 and 3, the laser branch (200) of the apparatus (10) for imaging and treatment of the target region of the skin is illustrated. As discussed above, and in some embodiments, the laser branch (200) is contained in the frame (20) of the apparatus (10) and has the laser path defined within the frame (20) of the apparatus (10). Further, the laser branch (200) may be configured to radiate electromagnetic rays along the laser path for treatment of the target region of the skin. In some embodiments, the laser branch (200) is configured to radiate electromagnetic rays, along the laser path, from the second end (24) of the frame (20) towards the first end (22) of the frame (20) for treatment of the target region of the skin. In some embodiments, the laser branch (200) of the apparatus (10) is adapted to radiate a circular beam of electromagnetic rays at the second end (24) of the frame (20). In some embodiments, the laser branch (200) is further adapted to convert the circular beam of electromagnetic rays into a polygonal beam of electromagnetic rays, and output the polygonal beam of electromagnetic rays at the first end (22) of the frame (20) for treatment of the target region of the skin of the subject. In some embodiments, the sapphire window assembly is adapted to receive backscattered illumination light from the target region of the skin in the imaging path, and project the polygonal beam of electromagnetic rays on the target region of the skin in the laser path.

Still referring to FIGS. 1 to 3, in some embodiments, the laser branch (200) comprises a fiber connection/guide (210), a laser lens assembly (220) and the beam combiner (110) in the laser path for radiation of electromagnetic rays and for treatment of the target region of the skin. In accordance with the present disclosure, the fiber connection/guide (210) is configured to be disposed at the second end (24) of the frame (20), as shown in FIGS. 1 and 2. In some embodiments, the fiber connection/guide (210) is configured to receive a beam of electromagnetic rays. In some embodiments, the electromagnetic rays have a circular shape from an external source and radiate the circular beam of electromagnetic rays in the laser path of the apparatus (10), at the second end (24) of the frame (20). The fiber connection/guide (210) may be embodied as a treatment laser unit comprising a high-power laser fiber input source. In some embodiments, the treatment laser unit is a laser delivery unit. In some embodiments, the treatment laser unit may be connected to a laser console with a fiber that may extend into the fiber connection/guide (210). The fiber connection/guide (210) or the treatment laser unit may have different parameters of use that include wavelength, spot size, fluence, pulse duration, and pulse rate. In one embodiment, the different parameters are determined by a cross-section of the fiber that is received in the fiber connection/guide (210).

In some embodiments, the laser branch (200) further comprises the laser lens assembly (220) that is disposed downstream of the fiber connection/guide (210) in a direction of electromagnetic rays. In some embodiments, the laser lens assembly (220) is configured to receive the circular beam of electromagnetic rays that is output from the fiber connection/guide (210) at the second end (24) of the frame (20), along the laser path. The laser lens assembly (220) is further adapted to convert the circular beam of electromagnetic rays into a polygonal beam of electromagnetic rays. In accordance with the present disclosure, the laser lens assembly (220) may comprise a static lens unit (230) and a dynamic lens unit (240) disposed upstream of the static lens unit (230) in the direction of electromagnetic rays. In some embodiments, the dynamic lens unit (240) is configured to move relative to the static lens unit (230) along the direction of electromagnetic rays. In some embodiments, the relative movement of the dynamic lens unit (240) with respect to the static lens unit (230) converts the circular beam of electromagnetic rays into the polygonal beam of electromagnetic rays. The conversion of the geometry and/or shape of the electromagnetic rays may be, inter alia, an effect of one or more of the following: a distance of separation between the dynamic lens unit (240) and the static lens unit (230), the resultant power of the dynamic lens unit (240), the resultant power of the static lens unit (230), and shapes/geometry of the dynamic lens unit (240) and the static lens unit (230). In some embodiments, the apparatus is a handpiece with at least one button (not shown) for a user to adjust lenses and/or fire the treatment laser. In some embodiments, the apparatus is part of a system having a screen (not shown) for viewing images and with touch control of the apparatus for a user to adjust lens distance and/or fire the treatment laser.

In some embodiments, the laser lens assembly (220) comprises, within at least one of the static and dynamic lens units, one or more micro lens arrays configured with a specific shape and responsible for the beam shape conversion. For example, the micro lens arrays can have a square or a hexagonal arrangement. The shape of the laser treatment spot on the target will depend correspondingly on the shape of the micro lens arrangement.

In some embodiments, the intensity profile of the shape of the output beam will have a “flat top” profile, independent of the input beam profile which may be gaussian.

Further, and in some embodiments, the laser lens assembly (220) of the laser branch (200) facilitates the laser branch (200) to generate a polygonal treatment spot, at the first end (22) of the frame (20), based on the polygonal beam of electromagnetic rays. Moreover, the laser lens assembly (220) described above facilitates the laser branch (200) to generate the polygonal treatment spot having varying size, based on one or more of the following: the distance of separation between the dynamic lens unit (240) and the static lens unit (230), the resultant power of the dynamic lens unit (240), the resultant power of the static lens unit (230), and shapes/geometry of the dynamic lens unit (240) and the static lens unit (230). In some embodiments, the laser branch is configured to generate the polygonal treatment spot having a rectangular, and more specifically square, shape. In some embodiments, the laser branch is configured to generate the polygonal treatment spot having a hexagonal shape.

In some embodiments, the laser lens assembly (220) further comprises a motor and nut-bracket assembly (250), as shown in FIGS. 1 and 2. The motor and nut-bracket assembly (250) may be configured to be operatively coupled with the dynamic lens unit (240) for controllably moving the dynamic lens unit (240) relative to the static lens unit (230), along the direction of electromagnetic rays. The motor and nut-bracket assembly (250) may comprise an electric motor (252) that, in some embodiments, are coupled to the frame (20) of the apparatus (10). The electric motor (252) may be driven by a power source, for example a battery, for actuating a nut-bracket (254), via a motor nut, for moving the dynamic lens unit (240) relative to the static lens unit (230). In some embodiments, the motor and nut-bracket assembly (250) are configured to move the dynamic lens unit (240) towards and away from the static lens unit (230) in a controlled manner, for converting the circular beam of electromagnetic rays into the polygonal beam of electromagnetic rays. The motor and nut-bracket assembly (250) may further comprise a sensor (256) for detecting and/or ensuring accuracy of the electric motor (252) for maintaining distance of separation between the dynamic lens unit (240) and the static lens unit (230), within the range of −/+0.2 mm, and eliminating the need of liner encoder for accuracy.

In some embodiments of the present disclosure, the static lens unit (230) is a combination of two or more lenses (232), for example micro lenses, mounted and/or arranged in a housing (234). Also, the dynamic lens unit (240) may be embodied as a combination of two or more lenses (242), for example micro lenses, mounted and/or arranged in a housing (244). Without deviating from the scope of the present disclosure, the housing (234) of the static lens unit (230) may comprise at least two guide pins (236) extending outwardly from the housing (234) of the static lens unit (230) towards the housing (244) of the dynamic lens unit (240). Further, said guide pins (236) may be received in the complimentary holes (246) defined in the housing (244) of the dynamic lens unit (240). In accordance with the present disclosure, said guide pins (236) and the holes (246) facilitate movement of the dynamic lens unit (240) relative to the static lens unit (230) along the direction of the laser path and the electromagnetic rays.

In some embodiments, there is a method wherein the user previews images obtained by the image branch of the apparatus. The user may then change the laser treatment parameters based on the image previews. The user may also change the treatment size as part of the treatment parameters by adjusting the distance between the static and dynamic lens.

In some embodiments, the laser branch (200) furthermore comprises the beam combiner (110) disposed downstream of the laser lens assembly (220) in the direction of the laser path. In some embodiments, the beam combiner (110) is configured to receive the polygonal beam of electromagnetic rays from the laser lens assembly (220), and to project the polygonal beam of electromagnetic radiation on the target region (to be treated) of the skin of the subject. Further, for the reason that the beam combiner (110) acts as a refractive medium for the electromagnetic rays, the beam combiner (110) projects the polygonal beam of electromagnetic rays on the target region of the skin and generates the polygonal treatment spot at the target region of the skin. As illustrated in the exemplary embodiment of FIGS. 1 and 2, the beam combiner (110) may be arranged inclined to the direction of electromagnetic rays. In some embodiments of the present disclosure, the beam combiner (110) projects the polygonal beam of electromagnetic rays on the target region, via the protective window (32) and the sapphire window assembly (30) at the first end (22) of the frame (20).

In some embodiments of the present disclosure, the laser branch (200) of the apparatus (10) may comprise a beam collimation assembly (260), as shown in FIGS. 1 and 2, arranged between the fiber guide (210) and the laser lens assembly (220). The beam collimation assembly (260) may be adapted to collimate the circular beam of electromagnetic rays and project the circular beam of electromagnetic rays on the laser lens assembly (220) for conversion into the polygonal beam of electromagnetic rays.

In accordance with the present disclosure, it can be contemplated that the beam combiner (110) is configured to refract the electromagnetic rays as well as reflect the collected illumination light. Also, in some embodiments, it can be contemplated that the sapphire window assembly (30) of the frame (20) is adapted to receive backscattered illumination light from the target region of the skin in the imaging path, and project the polygonal beam of electromagnetic rays on the target region of the skin in the laser path.

Without deviating from the scope of the present disclosure, the laser lens assembly (220) of the apparatus (10) eliminates the need of using a special and separate adapter for converting the circular beam of electromagnetic rays into the polygonal beam of electromagnetic rays, thereby reducing the complexity and simplifying the configuration/structure of the apparatus (10). Further, using an adapter, which is typically an element having a window with the desired geometry, such as a rectangular shape, may generate a static, and not dynamic, rectangular beam. If different polygonal shapes and/or sizes are desired, there will be a need for a set of different adapters, i.e. a single adapter for each shape. It can accordingly be contemplated that the apparatus (10) of the present disclosure facilitates better and quick treatment of the skin of the human.

In some embodiments, the laser branch is configured to provide a polygonal treatment beam having a uniform, homogeneous, laser intensity. In some embodiments, the laser branch is configured to provide a polygonal treatment beam having a non-uniform, inhomogeneous, laser intensity, e.g., a treatment beam with a predetermined intensity gradient.

In some embodiments, it is appreciated that the laser branch can also be adapted to provide a plurality of distinct, spaced-apart polygonal treatment beams and treatment spots, by dividing the input electromagnetic beams or the output electromagnetic beam. This can be achieved, for example, by including a plurality of the laser lens assembly (220), each receiving a respective circular beam and outputting a respective polygonal beam.

In some embodiments, the apparatus of the current disclosure is part of a skin diagnostic and treatment system using machine learning models. In some embodiments, the skin diagnostic and treatment system analyzes captured multi-spectral images to automatically determine the treatment parameters of the laser treatment of the apparatus. In some embodiments, the skin diagnostic and treatment system analyzes a set of multi-spectral images taken right after the laser treatment is fired to determine new treatment parameters.

It is to be understood that the aspects and embodiments of the disclosure described above may be used in any combination with each other. Several of the aspects and embodiments may be combined together to form a further embodiment of the disclosure.

The various embodiments of the present disclosure have been described above with reference to the accompanying drawings. The present disclosure is not limited to the illustrated embodiments; rather, these embodiments are intended to fully and completely disclose the subject matter of the disclosure to those skilled in this art. In the drawings, like numbers refer to like elements throughout. Thicknesses and dimensions of some components may be exaggerated for clarity.

Spatially relative terms, such as “under”, “below”, “lower”, “over”, “upper”, “top”, “bottom” and the like, may be used herein for ease of description to describe one element or feature's relationship to another element(s) or feature(s) as illustrated in the FIGS. It will be understood that the spatially relative terms are intended to encompass different orientations of the device in use or operation in addition to the orientation depicted in the FIGS. For example, if the device in the figures is turned over, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Herein, the terms “attached”, “connected”, “interconnected”, “contacting”, “mounted”, “coupled” and the like can mean either direct or indirect attachment or contact between elements, unless stated otherwise.

Well-known functions or constructions may not be described in detail for brevity and/or clarity. As used herein the expression “and/or” includes any and all combinations of one or more of the associated listed items.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises”, “comprising”, “includes” and/or “including” when used in this specification, specify the presence of stated features, operations, elements, and/or components, but do not preclude the presence or addition of one or more other features, operations, elements, components, and/or groups thereof.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

### REFERENCE NUMERALS

| PARTICULARS | REFERRAL NUMERAL |
|---|---|
| Apparatus | 10 |
| Frame | 20 |
| First end | 22 |
| Second end | 24 |
| Sapphire window assembly | 30 |
| Protective window | 32 |
| LED assembly | 40 |
| Imaging branch | 100 |
| Image detector assembly | 102 |
| Beam combiner | 110 |
| First mirror | 120 |
| Image lens assembly | 130 |
| Lens | 132 |
| Lens | 134 |
| Lens | 136 |
| Aperture | 138 |
| Second mirror | 140 |
| Polarizer unit in path of collected light | 150 |
| Polarizer unit in path of illuminating light | 152 |
| Laser branch | 200 |
| Fiber connection/guide | 210 |
| Laser lens assembly | 220 |
| Static lens unit | 230 |
| Lenses | 232 |
| Housing | 234 |
| Guide pins | 236 |
| Dynamic lens unit | 240 |
| Lenses | 242 |
| Housing | 244 |
| Holes | 246 |
| Motor and nut-bracket assembly | 250 |
| Electric motor | 252 |
| Nut bracket | 254 |
| Sensor | 256 |
| Beam collimation assembly | 260 |
| Longitudinal axis | X-X′ |

1-19. (canceled)

20. An apparatus for imaging and treatment of skin of a subject, the apparatus comprising:
a frame configured to circumscribe a target region of the skin of the subject, the frame having a first end and a second end opposite to the first end;
an imaging branch contained in the frame, the imaging branch having an imaging path and configured to generate an image of the target region of the skin that is in proximity to the first end of the frame; and
a laser branch contained in the frame, the laser branch having a laser path and configured to radiate electromagnetic rays from the second end of the frame towards the first end of the frame for treatment of the target region of the skin, wherein the laser branch is adapted to convert a shape of a beam of electromagnetic rays by:
radiating a circular beam of electromagnetic rays at the second end of the frame, and
outputting a polygonal beam of electromagnetic rays at the first end of the frame for treatment of the target region of the skin of the subject,
wherein the laser branch is configured to generate a corresponding polygonal treatment spot based on the polygonal beam of electromagnetic rays.

21. The apparatus as claimed in claim 20, wherein the laser branch is configured to generate the polygonal treatment spots having varying sizes or varying shapes, the varying shapes selected from: rectangular shape, square shape and hexagonal shape.

22. The apparatus as claimed in claim 20, wherein the shape of the output polygonal beam of electromagnetic rays has a flat top intensity profile, independent of a gaussian profile of the shape of the radiated circular beam of electromagnetic rays.

23. The apparatus as claimed in claim 20, wherein the laser branch is configured to provide a polygonal treatment beam having a non-uniform intensity profile with a predetermined intensity gradient across the polygonal treatment beam.

24. The apparatus as claimed in claim 20, wherein the laser branch comprises:
a fiber guide disposed at the second end of the frame, the fiber guide configured to radiate the circular beam of electromagnetic rays; and
a laser lens assembly disposed downstream of the fiber guide in a direction of electromagnetic rays, the laser lens assembly adapted to convert the circular beam of electromagnetic rays into the polygonal beam of electromagnetic rays.

25. The apparatus as claimed in claim 24, wherein the laser lens assembly comprises:
a static lens unit;
a dynamic lens unit disposed upstream of the static lens unit in the direction of electromagnetic rays, the dynamic lens unit configured to move relative to the static lens unit along the direction of electromagnetic rays; and
a motor and nut-bracket assembly operatively coupled with the dynamic lens unit for moving the dynamic lens unit relative to the static lens unit.

26. The apparatus as claimed in claim 25, wherein the laser lens assembly comprises, within at least one of the static lens unit and the dynamic lens unit, one or more micro lens arrays configured with a shape adapted for conversion of the shape of the beam of electromagnetic rays.

27. The apparatus of claim 25, wherein the motor and nut-bracket assembly are configured to controllably move the dynamic lens unit relative to the static lens unit, along direction of electromagnetic rays, such that movement of the dynamic lens unit effectuates a controlled conversion of the circular beam of electromagnetic rays into the polygonal beam of electromagnetic rays.

28. The apparatus as claimed in claim 20, wherein the frame comprises a sapphire window assembly at the first end thereof, the sapphire window assembly adapted to simultaneously:
receive backscattered illumination light from the target region of the skin, in the imaging path, and
project the polygonal beam of electromagnetic rays on the target region of the skin, in the laser path.

29. The apparatus as claimed in claim 20, wherein the laser branch is configured to provide a plurality of distinct, spaced-apart polygonal treatment beams, the plurality of distinct, spaced-apart polygonal treatment beams is generated by dividing an input circular beam of electromagnetic rays or dividing a polygonal output beam of electromagnetic rays.

30. The apparatus as claimed in claim 29, comprising a plurality of laser lens assemblies, each configured to receive a respective circular beam of electromagnetic rays and output a corresponding polygonal beam of electromagnetic rays.

31. The apparatus as claimed in claim 30, wherein each laser lens assembly is independently configurable to output polygonal beams of different shapes or sizes.

32. The apparatus as claimed in claim 20, further comprising a control system implementing one or more machine learning models configured to analyze images captured by the imaging branch, to automatically determine one or more updated treatment parameters for the laser branch.

33. The apparatus as claimed in claim 32, wherein the one or more updated treatment parameters are used to modify a subsequent laser pulse or treatment spot.

34. The apparatus as claimed in claim 33, wherein the one or more machine learning models are configured to perform real-time adjustment of at least one of: pulse energy, pulse duration, spot size, spot shape, or treatment pattern.

35. A method for imaging and treatment of skin of a subject using the apparatus of claim 1, the method comprising:
previewing, by a user, images of the target region of the skin generated by the imaging branch of the apparatus;
adjusting one or more laser treatment parameters of the laser branch based on the previewed images;
adjusting at least one of a size or a shape of a polygonal treatment spot by changing a distance between a static lens unit and a dynamic lens unit of a laser lens assembly; and
performing an imaging process or a treatment of the skin of the subject according to the adjusted laser treatment parameters and/or adjusted polygonal treatment spots.

36. The method of claim 35, wherein said adjusting the at least one of the size or the shape of the polygonal treatment spot comprises:
radiating a circular beam of electromagnetic rays at the second end of the frame;
outputting a polygonal beam of electromagnetic rays at the first end of the frame for treatment of the target region of the skin of the subject;
and performing the imaging process or the treatment of the skin of the subject according to the adjusted polygonal treatment spots.

37. The method of claim 35, wherein said adjusting the one or more laser treatment parameters comprises:
obtaining multi-spectral images of the target region of the skin of the subject;
analyzing the multi-spectral images to automatically determine the treatment parameters of the laser treatment of the apparatus; and
and performing the imaging process or the treatment of the skin of the subject according to the adjusted laser treatment parameters.

38. The method of claim 35, wherein said adjusting the one or more laser treatment parameters comprises applying machine learning models for skin diagnosis of the skin of the subject.

39. The method of claim 35, wherein said adjusting the at least one of the size or the shape of the polygonal treatment spot comprises applying machine learning models for determining.
